(19) United States
(12) Patent Application Publication
(10) Pub. No.: US 2008/0105866 A1
(43) Pub. Date: May 8, 2008

(54) Method of fabricating organic thin film transistor using self assembled monolayer-forming compound containing dichlorophosphoryl group

(75) Inventors: Eun Jeong Jeong, Yongin-si (KR); Jung Seok Hahn, Yongin-si (KR); Jeong Il Park, Yongin-si (KR); Sang Yoon Lee, Yongin-si (KR)

Correspondence Address: HARNESS, DICKEY & PIERCE, P.L.C., P.O. BOX 8910, RESTON, VA, 20195, US

(73) Assignee: SAMSUNG ELECTRONICS CO., LTD

(21) Appl. No.: 11/802,657

(22) Filed: May 24, 2007

(30) Foreign Application Priority Data
Nov. 2, 2006 (KR) 10-2006-0107677

(51) Int. Cl. H01L 51/40; C07D 333/02; C07F 9/08; H01L 29/08

(52) U.S. Cl. 257/40; 438/99; 549/6; 568/14; 257/E51.001; 257/E21.703

(57) ABSTRACT

Disclosed is a method of fabricating an organic thin film transistor including a substrate, a gate electrode, a gate insulating layer, metal oxide source/drain electrodes, and an organic semiconductor layer, in which the surface of the metal oxide source/drain electrodes or of the metal oxide source/drain electrodes and gate insulating layer is treated with a self assembled monolayer-forming compound containing a dichlorophosphoryl group. According to the method of example embodiments, the work function of the metal oxide of the source/drain electrodes may be increased to be higher than that with no SAM-forming electrode, thus making it possible to fabricate an improved organic thin film transistor having increased charge mobility.

### PRIORITY STATEMENT

This non-provisional application claims priority under U.S.C. § 119 to Korean Patent Application No. 2006-107677, filed on Nov. 02, 2006, in the Korean Intellectual Property Office (KIPO), the entire contents of which are herein incorporated by reference.

### BACKGROUND

1. Field

Example embodiments relate to a method of fabricating an organic thin film transistor (OTFT) using a self assembled monolayer (SAM)-forming compound containing a dichlorophosphoryl group. Other example embodiments relate to a method of fabricating an OTFT including a substrate, a gate electrode, a gate insulating layer, metal oxide source/drain electrodes, and an organic semiconductor layer, in which the surface of the metal oxide source/drain electrodes or of the metal oxide source/drain electrodes and gate insulating layer is treated with an SAM-forming compound containing a dichlorophosphoryl group, so that the work function of the metal oxide of the source/drain electrodes is increased to be higher than that with no SAM-forming electrode, therefore increasing charge mobility, and to an OTFT having increased charge mobility, fabricated using the method.

2. Description of the Related Art

After the development of polyacetylene, which is a conjugated organic polymer having semiconductor properties, organic semiconductors have started receiving attention as an electric and electronic material thanks to the advantages of organic material, for example, the variety of synthesis methods, relatively easy formability into fibers or films, flexibility, conductivity, and decreased preparation costs, and thus have been intensively and extensively studied in the wide field of functional electronic devices and optical devices.

Among devices using such a conductive polymer, research into OTFTs using organic material as an active layer is being conducted all over the world these days. The OTFT may have a structure similar to a conventional Si TFT, with the exception that the semiconductor region thereof is formed using an organic material, instead of Si. Compared to conventional Si TFTs, OTFTs may be advantageous because a semiconductor layer may be formed through a printing process under atmospheric pressure in place of plasma-enhanced chemical vapor deposition, and all of the fabrication processes may be carried out through a roll-to-roll process using a plastic substrate, if necessary, thereby decreasing the cost of fabricating the transistor. Accordingly, the OTFT may be expected to be variously applicable to devices for driving active displays, smart cards and/or plastic chips for inventory tags.

The electrode of an OTFT may be formed with gold (Au) metal. Because gold (Au) has a work function similar to an organic semiconductor, improved organic semiconductor properties may be manifested, but may have undesirable processibility when compared with other metals. Thus, recent attempts have been made to use metal oxides, for example, indium tin oxide (ITO), in order to achieve decreased production costs upon application to mass production lines. However, metal oxides, for example, ITO, have a work function different from that of the organic semiconductor, and therefore a Schottky barrier is formed between the metal oxide electrode and the organic semiconductor layer, undesirably resulting in increased on-current drop.

With the goal of solving problems, surface treatment techniques for forming an SAM on the metal oxide electrode have been conventionally employed to minimize or decrease the barrier of the two interfaces and decrease contact resistance. The SAM technique, which is a process of treating the surface of metal oxide or the surface of the organic material with organic material, may be applied to surface treatment of OTFTs or OELDs. Because most of the insulating layer or electrodes of the OTFT are formed of metal oxide, the above technique may decrease the contact resistance with the upper organic semiconductor.

In this regard, conventional techniques using RSO₃H, phosphoric acid, or phosphonic acid as an SAM compound have been proposed. However, as illustrated in FIG. 1, because the above material is formed into a film on metal oxide through physical adsorption, it may undesirably be dissociated from the metal oxide at increased temperatures or in an increased vacuum. Ultimately, there is a need for an SAM material which is able to induce a chemical covalent bond which is more stable.

Typically representative of covalently bondable SAM material is trichlorosilane. However, this material may self-oligomerize due to moisture present in the air, and thus, may be difficult to apply to mass production lines. As another covalently bondable SAM material, a compound in which a monochlorophosphoryl group is introduced to the side chain of a polymer, may suffer because it does not play a sufficient role in forming the SAM attributable to the presence of only one reactive group.

### SUMMARY

Accordingly, example embodiments have been made keeping in mind the above problems occurring in the related art, and example embodiments provide a method of fabricating an OTFT, in which the surface of the metal oxide source/drain electrodes or the surface of the metal oxide source/drain electrodes and gate electrode is treated with an SAM-forming compound containing a dichlorophosphoryl group able to form a covalent bond with metal oxide, so that the work function of the metal oxide is higher than that of the organic semiconductor material, thus resulting in improved electrical properties, including increased charge mobility. Example embodiments provide an OTFT, fabricated using the method. Example embodiments provide a display device, fabricated using the OTFT.

According to example embodiments, a method of fabricating an OTFT may include a substrate, a gate electrode, a gate insulating layer, metal oxide source/drain electrodes, and an organic semiconductor layer, the method comprising treating the surface of the metal oxide source/drain electrodes or the surface of the metal oxide source/drain electrodes and gate insulating layer with an SAM-forming compound containing a dichlorophosphoryl group.

Also, the method of example embodiments may further include annealing the surface of the metal oxide source/drain electrodes after treatment using the SAM-forming compound. Also, used in the method of example embodiments, the SAM-forming compound may further contain fluorine. Example embodiments provide an OTFT, fabricated using the above method. In addition, example embodiments provide a display device, fabricated using the OTFT.

### BRIEF DESCRIPTION OF THE DRAWINGS

Example embodiments will be more clearly understood from the following detailed description taken in conjunction with the accompanying drawings. FIGS. 1-5 represent non-limiting, example embodiments as described herein.

FIG. 1 is a schematic view illustrating the SAM which is physically adsorbed on the surface of metal oxide source/drain electrodes through treatment using an SAM-forming compound containing a sulfonic acid group, according to a conventional technique;

FIG. 2 is a schematic sectional view illustrating the bottom-contact type OTFT according to example embodiments;

FIG. 3 is a schematic sectional view illustrating the top-gate type OTFT according to example embodiments;

FIG. 4 is a schematic view illustrating the SAM which is covalently bonded to the surface of metal oxide source/drain electrodes through treatment using an SAM-forming compound containing a dichlorophosphoryl group, according to example embodiments; and

FIG. 5 is a graph illustrating the current transfer properties of the OTFTs of Examples 1 and 2 and Comparative Example 1.

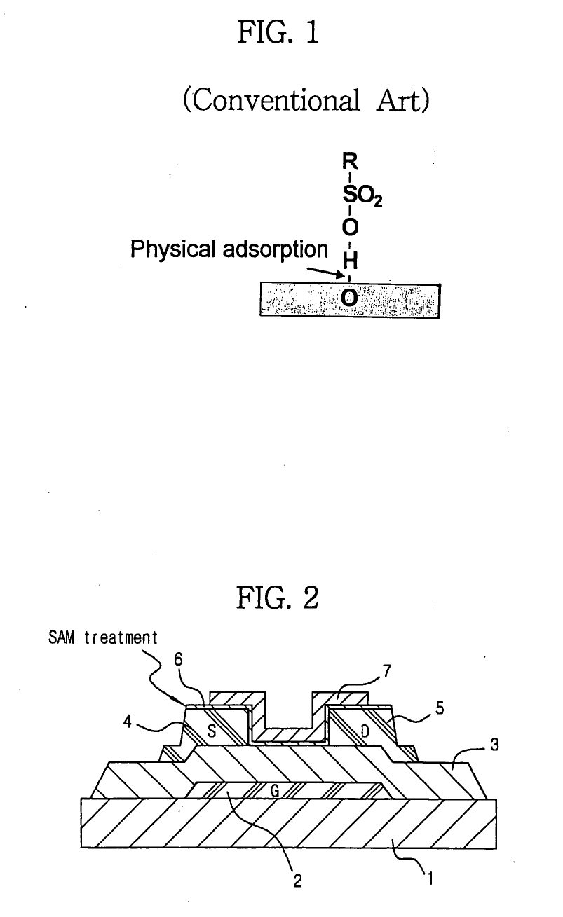

It should be noted that these Figures are intended to illustrate the general characteristics of methods, structure and/or materials utilized in certain example embodiments and to supplement the written description provided below. These drawings are not, however, to scale and may not precisely reflect the precise structural or performance characteristics of any given embodiment, and should not be interpreted as defining or limiting the range of values or properties encompassed by example embodiments. In particular, the relative thicknesses and positioning of molecules, layers, regions and/or structural elements may be reduced or exaggerated for clarity. The use of similar or identical reference numbers in the various drawings is intended to indicate the presence of a similar or identical element or feature.

### DETAILED DESCRIPTION OF EXAMPLE EMBODIMENTS

Hereinafter, a detailed description will be given of example embodiments with reference to the appended drawings. In the drawings, the thicknesses and widths of layers are exaggerated for clarity. Example embodiments may, however, be embodied in many different forms and should not be construed as limited to the example embodiments set forth herein. Rather, these example embodiments are provided so that this disclosure will be thorough and complete, and will fully convey the scope of example embodiments to those skilled in the art.

It will be understood that when an element or layer is referred to as being “on”, “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numbers refer to like elements throughout. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of example embodiments.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Example embodiments are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments should not be construed as limited to the particular shapes of regions illustrated herein but are to include deviations in shapes that result, for example, from manufacturing. For example, an implanted region illustrated as a rectangle will, typically, have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of example embodiments.

Unless otherwise defined, all terms (including technical and scientific terms) used herein have the same meaning as commonly understood by one of ordinary skill in the art to which example embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

According to example embodiments, the method of fabricating an OTFT may include a substrate, a gate electrode, a gate insulating layer, metal oxide source/drain electrodes, and an organic semiconductor layer, wherein the surface of the metal oxide source/drain electrodes or the surface of the metal oxide source/drain electrodes and gate insulating layer is treated with an SAM-forming compound containing a dichlorophosphoryl group.

The structure of OTFT fabricated through the method of example embodiments may have a bottom contact type or a top gate type, but example embodiments may not be limited thereto. For example, the OTFT fabrication method of example embodiments may include forming a gate electrode on a substrate, forming a gate insulating layer on the gate electrode, forming metal oxide source/drain electrodes on the gate insulating layer, forming an SAM on the surface of the metal oxide source/drain electrodes, and forming an organic semiconductor layer on the insulating layer and the metal oxide source/drain electrodes, resulting in a bottom contact type device, or alternatively, forming metal oxide source/drain electrodes on a substrate, forming an SAM on the surface of the metal oxide source/drain electrodes, forming an organic semiconductor layer between the metal oxide source/drain electrodes, forming an insulating layer on the organic semiconductor layer, and forming a gate electrode on the insulating layer, resulting in a top gate type device.

Below, the fabrication method of example embodiments is stepwisely described. The following procedure is based on the method of fabricating a bottom contact type OTFT, but the fabrication method of example embodiments may also be applied to a top gate type OTFT.

A substrate may be washed through a typical process to remove impurities, and then a gate electrode may be formed thereon through deposition and patterning. Examples of material for the substrate may include, but may not be limited to, glass, silicon, and plastic.

The material for the gate electrode may include a typical metal or conductive polymer, specific examples thereof including, but not being limited to, gold (Au), silver (Ag), aluminum (Al), nickel (Ni), molybdenum (Mo), tungsten (W), indium tin oxide (ITO), polythiophene, polyaniline, polyacetylene, polypyrrole, polyphenylene vinylene, and a mixture of PEDOT (polyethylenedioxythiophene) and PSS (polystyrenesulfonate).

After the gate electrode is formed on the substrate, a gate insulating layer may be formed on the gate electrode through a typical process. Examples of the material for the gate insulating layer may include, but may not be limited to, organic material containing oxygen, for example, PVP, polyimide, PVA, PE, siloxane, organic-inorganic hybrids, and derivatives thereof, and inorganic material, for example, SiNₓ (0<x<4), SiO₂ and Al₂O₃. The thickness thereof may be appropriately controlled depending on need.

Using an insulator including a cross-linking agent and an organic-inorganic hybrid type insulator, the gate insulating layer may be formed to a thickness of about 3000 Å˜about 7000 Å. Although the process of forming the gate insulating layer may not be particularly limited, a vacuum deposition process or a solution process may be applied.

Soft baking may be performed at about 60° C.˜about 150° C. for about 1 min about 10 min and then hard baking may be performed at about 100° C.˜200° C. for about 0.5 hours˜about 3 hours, if necessary.

After the gate insulating layer is formed, metal oxide source/drain electrodes may be formed thereon. Metal oxide may be formed into a thin film on the gate insulating layer through a typical thin film formation process, after which the thin film is subjected to typical exposure and development to expose the region with source/drain electrodes (or regions other than source/drain electrodes), followed by performing a typical etching process using an acidic aqueous solution and then finally removing a photoresist using a photoresist stripper, thereby forming the metal oxide source/drain electrodes.

As such, examples of the metal oxide for source/drain electrodes may include, but may not be limited to, ITO (indium tin oxide) and IZO (indium zinc oxide). Examples of the thin film formation process for forming metal oxide on the gate insulating layer may include, but may not be limited to, thermal evaporation, spin coating, roll coating, spray coating, and printing.

After the metal oxide source/drain electrodes are formed, the surface of the metal oxide source/drain electrodes may be treated with an SAM-forming compound, containing a dichlorophosphoryl group, under predetermined or given conditions.

The SAM-forming compound may contain a dichlorophosphoryl group (—POCl₂) functioning as a binding group to the surface of the metal oxide source/drain electrodes in order to form a chemical covalent bond with the metal oxide. For example, as illustrated in FIG. 4, the dichlorophosphoryl group may be covalently bonded with oxygen of the substrate to thus form a covalent bond via a —PO bond to the surface of metal oxide while emitting HCl, leading to an SAM which is more stable than in the case of physical adsorption.

Although the SAM-forming compound containing a dichlorophosphoryl group may not be particularly limited, it is represented by Formula 1 below:

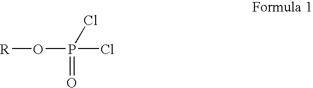

wherein R is selected from the group consisting of hydrogen, halogen, a substituted or unsubstituted C₆-C₃₀ aromatic group, a substituted or unsubstituted C₅-C₃₀ heteroaromatic group containing one or more hetero atoms, a substituted or unsubstituted C₁-C₂₀ alkyl group, and a substituted or unsubstituted C₁-C₂₀ alkyl group containing at least one selected from among

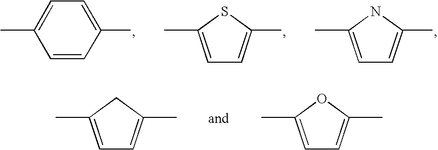

in the main chain thereof, in which the substituent is one or more selected from the group consisting of halogen, an unsubstituted or halogen-substituted C₁-C₁₂ alkyl group, an alkoxy group, an ether group, a carboxyl group, a thiol group, and an amine group.

As such, the compound of Formula 1 may be synthesized via a typical synthesis process, and for example, may be synthesized according to Reaction 1 below:

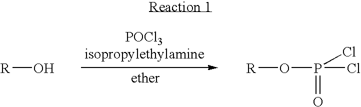

wherein R is as defined in Formula 1.

Examples of the SAM-forming compound containing a dichlorophosphoryl group of Formula 1 may include, but may not be limited to, compounds represented by Formulas 2 to 4 below:

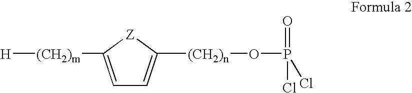

wherein Z is sulfur, nitrogen, carbon or oxygen, m is an integer from about 0 to about 20, and n is an integer from about 0 to about 20;

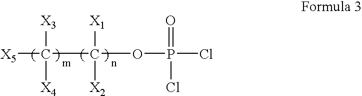

wherein X₁, X₂, X₃, X₄, and X₅ are each independently hydrogen, halogen, or a C₁-C₃₀ alkyl group substituted with one or more selected from among hydrogen, halogen, oxygen and nitrogen, and

m is an integer from about 1 to about 20, and n is an integer from about 0 to about 20; and

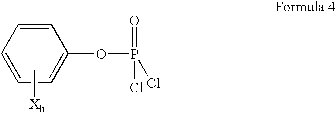

wherein X is hydrogen, halogen or CF₃, and h is an integer from about 1 to about 5.

Examples of the SAM-forming compound containing the dichlorophosphoryl group of Formulas 2 to 4 may include, but may not be limited to, compounds represented by Formulas 5 to 15 below:

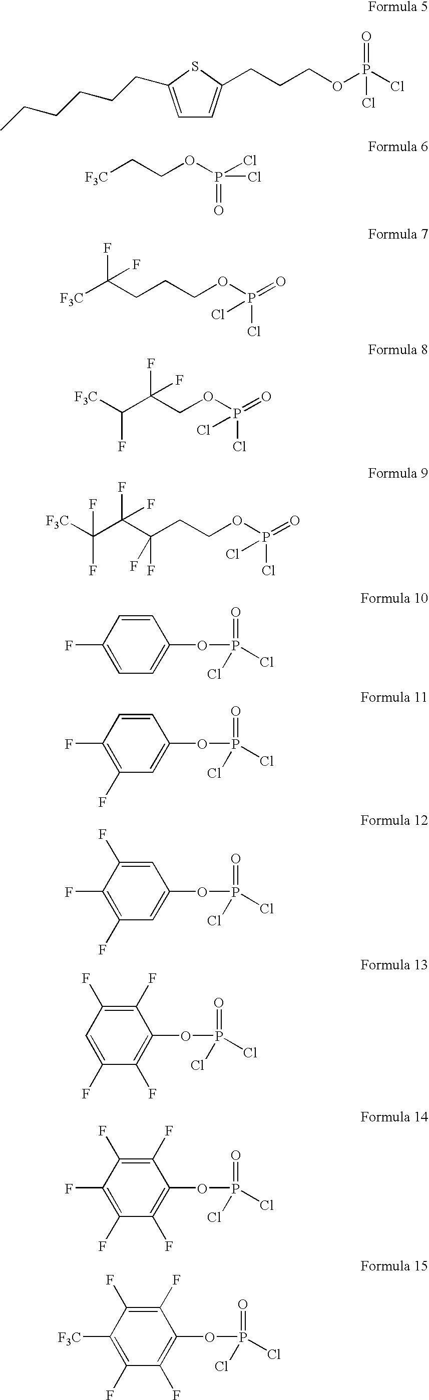

The SAM-forming compound containing a dichlorophosphoryl group may include halogen as a substituent, the halogen including one or more fluorine atoms. Although the exact mechanism has not yet been found, it is assumed that when the surface of the metal oxide source/drain electrodes is treated with the SAM-forming compound containing fluorine, fluorine contained in the SAM may function to enable electrons to be more easily received from the organic semiconductor material to thus generate a hole-doping effect, so that the work function of the metal oxide of the source/drain electrodes may increase to be higher than that with no SAM-forming electrode, resulting in improved charge mobility.

The SAM-forming compound may be bonded with the gate insulating layer in contact with the organic semiconductor layer, as well as the metal oxide. Therefore, the SAM-forming compound may be used for the surface treatment of the gate insulating layer, in addition to the surface of the metal oxide. The gate insulating layer may be formed of oxygen-containing organic or inorganic material in order to cause a covalent bond.

In example embodiments, the SAM treatment of the surface of the metal oxide source/drain electrodes may be performed using a solution obtained by dissolving the SAM-forming compound containing a dichlorophosphoryl group in a solvent selected from an organic solvent, and mixtures thereof. For example, the surface treatment of the metal oxide source/drain electrodes may be carried out by immersing the surface of the metal oxide source/drain electrodes in an SAM-forming solution at a predetermined or given temperature for a predetermined or given time period. Examples of the organic solvent may include, but may not be limited to, alcohols, ethers, chlorine-based alkanes, aromatics, and glycols.

In the SAM-forming solution, the SAM-forming compound containing a dichlorophosphoryl group may be included in an appropriate amount, for example, about 0.001 wt %˜about 50 wt %. Moreover, the SAM surface treatment of the metal oxide source/drain electrodes may be performed without limitation under typical conditions, and specifically may be conducted at about 0° C.˜about 150° C. for a time period ranging from about 10 sec to about 30 min through dipping, spin coating, or jetting.

Finally, on the metal oxide source/drain electrodes, an organic semiconductor material may be applied through a typical coating process, thus forming an organic semiconductor layer. Examples of the organic semiconductor material may include, but may not be limited to, pentacene, tetracene, copper phthalocyanine, polythiophene, polyaniline, polyacetylene, polypyrrole, polyphenylene vinylene, and derivatives thereof, and also, examples of the coating process for deposition thereof may include, but may not be limited to, thermal evaporation, screen printing, printing, spin coating, dip coating and/or ink jetting.

The method of fabricating the OTFT according to example embodiments may further include annealing the surface of the metal oxide source/drain electrodes, after surface treatment using the SAM-forming compound.

When the surface of the metal oxide source/drain electrodes is treated and then annealed, the strength of adhesion of the source/drain electrodes may increase, therefore making it possible to fabricate a transistor having higher charge mobility than when performing only surface treatment using the SAM-forming compound. The annealing process may not be particularly limited, but may be performed at about 20° C.˜about 200° C. for a time period ranging from about 10 min to about 2 hours.

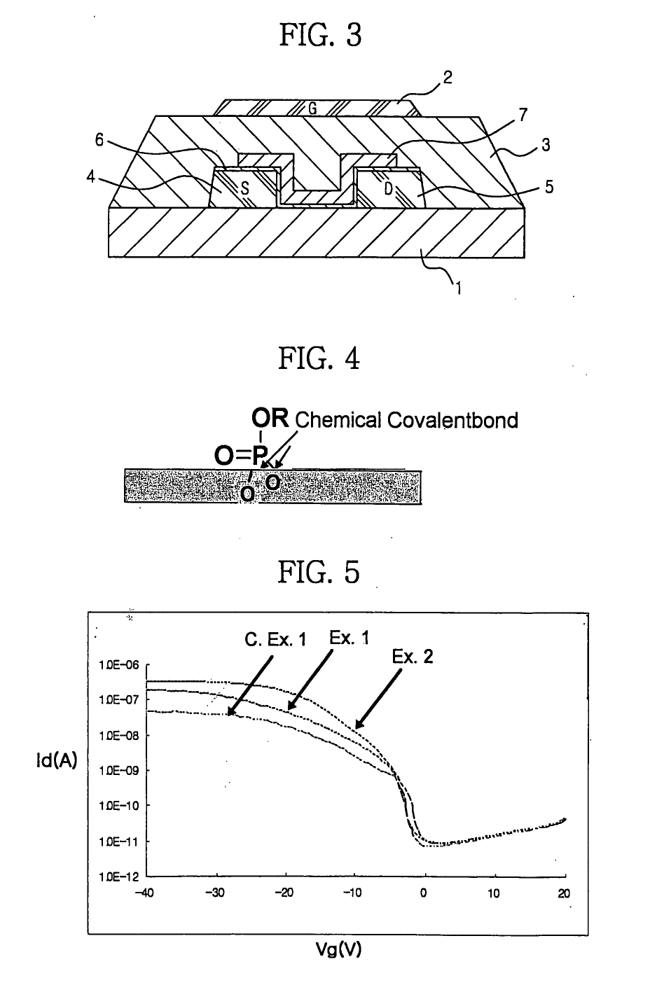

According to example embodiments, the OTFT thus fabricated may be a bottom contact type OTFT, comprising a gate electrode 2, a gate insulating layer 3, metal oxide source/drain electrodes 4, 5, an SAM 6, and an organic semiconductor layer 7, which are sequentially formed on a substrate 1, as illustrated in FIG. 2. The OTFT of example embodiments may also be a top gate type OTFT, comprising metal oxide source/drain electrodes 4, 5, an SAM 6, an organic semiconductor layer 7, a gate insulating layer 3, and a gate electrode 2, which are sequentially formed on a substrate 1, as illustrated in FIG. 3.

In the OTFT of example embodiments, as the work function of the metal oxide of the source/drain electrodes is higher than that with no SAM-forming electrode, improved electrical properties, for example, increased charge mobility, may be realized.

Therefore, the OTFT of example embodiments may be effectively applied to display devices, for example, electroluminescent devices, liquid crystal devices and/or electrophoretic devices.

A better understanding of example embodiments may be obtained in light of the following examples which are set forth to illustrate, but are not to be construed to limit example embodiments.

### EXAMPLE 1

On a washed glass substrate, a gate electrode was formed to a thickness of about 1500 Å through sputtering using Al/Nd. Subsequently, a PVP-based insulator was applied to a thickness of about 5000 Å on the gate electrode through spin coating at about 1000 rpm to thus form a gate insulating layer, which was then subjected to soft baking at about 100° C. for about 5 min, resulting in a desired gate insulating layer. ITO was deposited to a thickness of about 1000 Å thereon through thermal evaporation under a vacuum condition (about 2×10⁻⁷ torr, substrate temperature of about 50° C., and deposition rate of about 0.85 Å/sec), followed by performing photolithography to thus form an ITO electrode pattern. Thereafter, the ITO electrode was immersed in an SAM-forming solution, obtained by dissolving an SAM-forming compound represented by Formula 16 below in about 0.2 wt % ethyl alcohol, at room temperature for about 10 sec to thus realize surface treatment, and was then annealed for about 30 min to thus evaporate the solvent. Then, polythiophene was applied to a thickness of about 800 Å through spin coating at about 1000 rpm, thereby fabricating the OTFT.

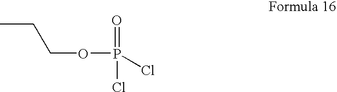

### EXAMPLE 2

An OTFT was fabricated in the same manner as in Example 1, with the exception that a compound containing a dichlorophosphoryl group having fluorine, represented by Formula 17 below, was used as the SAM-forming compound:

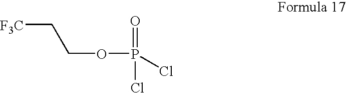

### COMPARATIVE EXAMPLE 1

An OTFT was fabricated in the same manner as in Example 1, with the exception that the ITO electrode was surface treated without a SAM-forming compound.

In order to evaluate the change in properties of the source/drain electrodes and gate insulating layer through surface treatment using the SAM-forming compound according to example embodiments, the current transfer properties of the OTFTs fabricated in Examples 1 and 2 and Comparative Example 1 were measured using a semiconductor analyzer (4200-SCS), available from KEITHLEY. The results are shown in FIG. 5.

The charge mobility, on-current (Iₒₙ), off-current (I_{off}), on/off ratio, and threshold voltage (Vₜₕ and Vₛₛ) of the OTFTs fabricated in Examples 1 and 2 and Comparative Example 1 were measured as follows. The results are shown in Table 1 below.
Among the electrical properties of the OTFTs fabricated in Examples 1 and 2 and Comparative Example 1, the charge mobility was calculated using the following current equation for the saturation region. For example, the current equation for the saturation region was converted into a graph of (I_{SD})^{1/2} and V_{G}, and the charge mobility was calculated from the slope of the converted graph:

$${I}_{SD} = \frac{{WC}_{0}}{2 L} {μ ( {V}_{G} - {V}_{T} )}^{2}$$

$$\sqrt{{I}_{SD}} = \sqrt{\frac{μ {C}_{0} W}{2 L}} ( {V}_{G} - {V}_{T} )$$

$$slope = \sqrt{\frac{μ {C}_{0} W}{2 L}}$$

$${μ}_{FET} = {( slope )}^{2} \frac{2 L}{{C}_{0} W}$$

wherein I_{SD} is source-drain current, μ or μ_{FET} is charge mobility, Cₒ is oxide film capacitance, W is the channel width, L is the channel length, V_{G} is the gate voltage, and V_{T} is the threshold voltage.

On-current (Iₒₙ) was determined to be the maximum current.
I_{off}, which is current flowing in the off-state, was determined to be the minimum current in the off-state.
On/Off ratio was determined as the ratio of the maximum current in the on-state to the minimum current in the off-state.

TABLE 1

| | Charge | | | | | |
|---|---|---|---|---|---|---|
| | Mobility | Iₒₙ (A) | I_{off} (A) | On/Off Ratio | Vₜₕ | Vₛₛ |
| C. Ex. 1 | 0.0036 | 4.50 × 10⁻⁸ | 7.30 × 10⁻¹² | 6.16 × 10⁻³ | −3 | −3 |
| Ex. 1 | 0.011 | 2.00 × 10⁻⁷ | 8.40 × 10⁻¹² | 2.38 × 10⁻⁴ | −4 | −5 |
| Ex. 2 | 0.046 | 3.40 × 10⁻⁷ | 9.10 × 10⁻¹² | 3.74 × 10⁻⁴ | −7 | −3 |

The examples treated with the SAM-forming compound according to example embodiments may charge mobility increased at least about 100 times while maintaining the leakage current (I_{off}), compared to the untreated example.

As is apparent from the results of Table 1, the OTFTs of example embodiments may have improved electrical properties, including charge mobility.

As described hereinbefore, example embodiments provide a method of fabricating an OTFT using an SAM-forming compound containing a dichlorophosphoryl group. According to the OTFT fabrication method of example embodiments, the work function of the metal oxide of source/drain electrodes may be increased to be higher than that of organic semiconductor material, thereby fabricating an improved OTFT having increased charge mobility.

Although example embodiments have been disclosed for illustrative purposes, those skilled in the art will appreciate that various modifications, additions and substitutions are possible, without departing from the scope and spirit of the accompanying claims.

What is claimed is:

1. A method comprising:
treating a surface of metal oxide source/drain electrodes or a surface of the metal oxide source/drain electrodes and a gate insulating layer with a self assembled monolayer-forming compound containing a dichlorophosphoryl group.

2. The method as set forth in claim 1, wherein the metal oxide source/drain electrodes and the gate insulating layer are components of an organic thin film transistor that further comprises a substrate, a gate electrode, and an organic semiconductor layer.

3. The method as set forth in claim 1, wherein the self assembled monolayer-forming compound containing a dichlorophosphoryl group is represented by Formula 1 below: 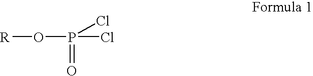
wherein R is selected from the group consisting of hydrogen, halogen, a substituted or unsubstituted C₆-C₃₀ aromatic group, a substituted or unsubstituted C₅-C₃₀ heteroaromatic group containing one or more hetero atoms, a substituted or unsubstituted C₁-C₂₀ alkyl group, and a substituted or unsubstituted C₁-C₂₀ alkyl group containing at least one selected from among 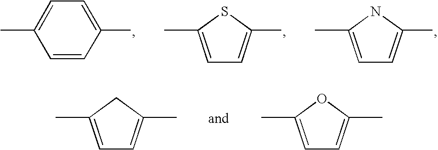
in a main chain thereof, in which a substituent is one or more selected from the group consisting of halogen, an unsubstituted or halogen-substituted C₁-C₁₂ alkyl group, an alkoxy group, an ether group, a carboxyl group, a thiol group, and an amine group.

4. The method as set forth in claim 3, wherein the self assembled monolayer-forming compound containing a dichlorophosphoryl group represented by Formula 1 is represented by Formulas 2 to 4 below: 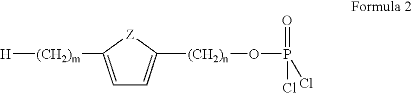
wherein Z is sulfur, nitrogen, carbon or oxygen, m is an integer from about 0 to about 20, and n is an integer from about 0 to about 20; 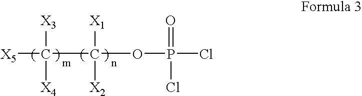
wherein X₁, X₂, X₃, X₄, and X₅ are each independently hydrogen, halogen, or a C₁-C₃₀ alkyl group substituted with one or more selected from among hydrogen, halogen, oxygen and nitrogen, and
m is an integer from about 1 to about 20, and n is an integer from about 0 to about 20; and 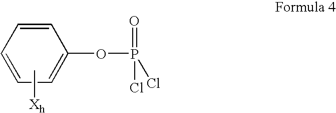
wherein X is hydrogen, halogen or CF₃, and h is an integer from about 1 to about 5.

5. The method as set forth in claim 4, wherein the self assembled monolayer-forming compound containing a dichlorophosphoryl group represented by Formulas 2 to 4 is represented by Formulas 5 to 15 below:

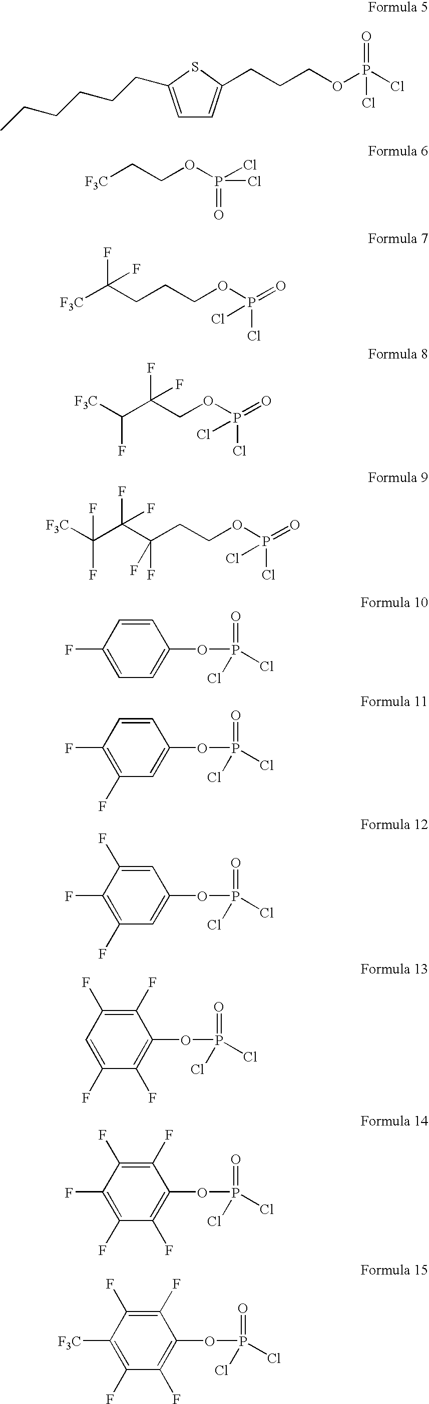

6. The method as set forth in claim 3, wherein the R of Formula 1 includes one or more fluorine atoms.

7. The method as set forth in claim 1, wherein treating is performed through an immersion process using a self assembled monolayer-forming solution obtained by dissolving the self assembled monolayer-forming compound containing a dichlorophosphoryl group in a solvent selected from an organic solvent, and mixtures thereof.

8. The method as set forth in claim 7, wherein the organic solvent is selected from the group consisting of alcohols, ethers, chlorine-based alkanes, aromatics, and glycols.

9. The method as set forth in claim 7, wherein the immersion process is performed at about 20° C.˜about 200° C. for a time period ranging from about 10 sec to about 2 hours.

10. The method as set forth in claim 1, further comprising:
annealing the surface of a metal oxide layer after treating the surface of the metal oxide layer.

11. The method as set forth in claim 2, wherein the substrate is formed using material selected from the group consisting of glass, silicon, and plastic.

12. The method as set forth in claim 2, wherein the gate electrode is formed using material selected from the group consisting of gold (Au), silver (Ag), aluminum (Al), nickel (Ni), molybdenum (Mo), tungsten (W), indium tin oxide (ITO), polythiophene, polyaniline, polyacetylene, polypyrrole, polyphenylene vinylene, and a mixture of PEDOT (polyethylenedioxythiophene) and PSS (polystyrenesulfonate).

13. The method as set forth in claim 1, wherein the gate insulating layer is formed using material selected from the group consisting of organic materials, including PVP, polyimide, PVA, PE, siloxane, organic-inorganic hybrids, and derivatives thereof, and inorganic materials, including SiNₓ (0<x<4), SiO₂ and Al₂O₃.

14. The method as set forth in claim 1, wherein the metal oxide source/drain electrodes are formed using material selected from the group consisting of ITO and IZO.

15. The method as set forth in claim 2, wherein the organic semiconductor layer is formed using material selected from the group consisting of pentacene, tetracene, copper phthalocyanine, polythiophene, polyaniline, polyacetylene, polypyrrole, polyphenylenevinylene, and derivatives thereof.

16. An organic thin film transistor, fabricated using the method of claim 1.

17. A display device, fabricated using the organic thin film transistor of claim 16.

18. A self assembled monolayer-forming compound comprising:
a dichlorophosphoryl group is represented by Formula 1 below: 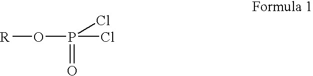
wherein R is selected from the group consisting of hydrogen, halogen, a substituted or unsubstituted C₆-C₃₀ aromatic group, a substituted or unsubstituted C₅-C₃₀ heteroaromatic group containing one or more hetero atoms, a substituted or unsubstituted C₁-C₂₀ alkyl group, and a substituted or unsubstituted C₁-C₂₀ alkyl group containing at least one selected from among 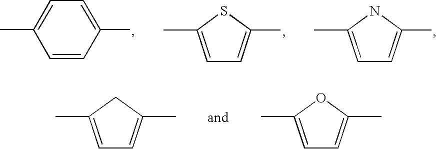
in a main chain thereof, in which a substituent is one or more selected from the group consisting of halogen, an unsubstituted or halogen-substituted C₁-C₁₂ alkyl group, an alkoxy group, an ether group, a carboxyl group, a thiol group, and an amine group.

19. The self assembled monolayer-forming compound as set forth in claim 18, wherein the self assembled monolayer-forming compound including a dichlorophosphoryl group represented by Formula 1 is represented by Formulas 2 to 4 below: 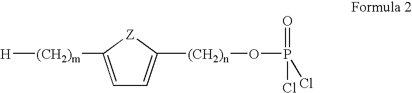
wherein Z is sulfur, nitrogen, carbon or oxygen, m is an integer from about 0 to about 20, and n is an integer from about 0 to about 20; 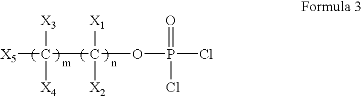
wherein X₁, X₂, X₃, X₄, and X₅ are each independently hydrogen, halogen, or a C₁-C₃₀ alkyl group substituted with one or more selected from among hydrogen, halogen, oxygen and nitrogen, and
m is an integer from about 1 to about 20, and n is an integer from about 0 to about 20; and 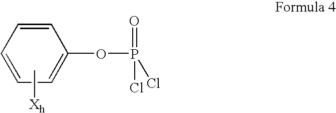
wherein X is hydrogen, halogen or CF₃, and h is an integer from about 1 to about 5.

20. The self assembled monolayer-forming compound as set forth in claim 19, wherein the self assembled monolayer-forming compound including a dichlorophosphoryl group represented by Formulas 2 to 4 is represented by Formulas 5 to 15 below:

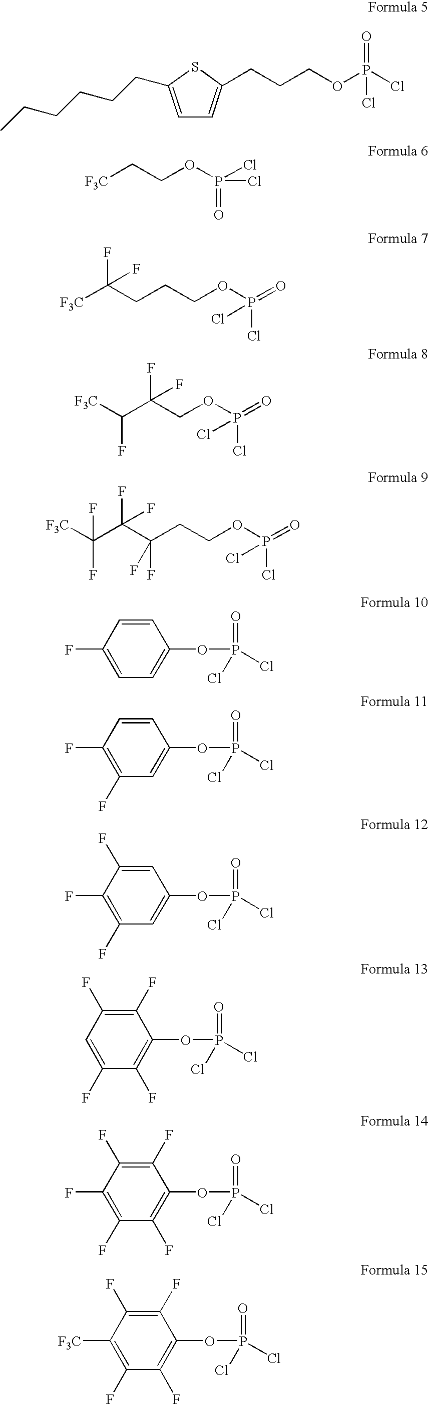

21. The self assembled monolayer-forming compound as set forth in claim 19, wherein the R of Formula 1 includes one or more fluorine atoms.
